(19) United States
(12) Patent Application Publication
(10) Pub. No.: US 2025/0099626 A1
(43) Pub. Date: Mar. 27, 2025

(54) COMPOSITIONS AND METHODS FOR OXONOL DYES WITH NEAR INFRARED FLUORESCENCE

(71) Applicants: Vladimir Birman, St. Louis, MO (US); Mikhail Berezin, St. Louis, MO (US); Isaac Fuentes, St. Louis, MO (US); Rui Tang, St. Louis, MO (US)

(72) Inventors: Vladimir Birman, St. Louis, MO (US); Mikhail Berezin, St. Louis, MO (US); Isaac Fuentes, St. Louis, MO (US); Rui Tang, St. Louis, MO (US)

(73) Assignee: Washington University, St. Louis, MO (US)

(21) Appl. No.: 18/891,309

(22) Filed: Sep. 20, 2024

Related U.S. Application Data
(60) Provisional application No. 63/584,849, filed on Sep. 22, 2023.

(51) Int. Cl. A61K 49/00

(52) U.S. Cl. CPC A61K 49/0039

(57) ABSTRACT

The present disclosure is directed to near infrared fluorescent oxonol dyes and imaging agent compositions including compounds according to Formula (I)

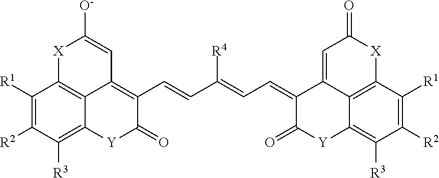

and their uses in bioimaging applications. The disclosure is further directed to oxonol precursor compositions and compounds according to Formula (II)

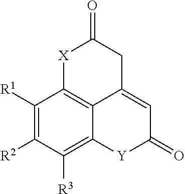

and their uses in synthesizing oxonol dyes and imaging agents.

### CROSS-REFERENCE TO RELATED APPLICATIONS

This application claims the benefit of priority to U.S. Provisional Application Ser. No. 63/584,849 filed on 22 Sep. 2023, which is incorporated herein by reference in its entirety.

### STATEMENT REGARDING FEDERALLY SPONSORED RESEARCH OR DEVELOPMENT

Not applicable.

### MATERIAL INCORPORATED-BY-REFERENCE

Not applicable.

### FIELD

The present disclosure generally relates to compositions comprising a new class of fluorescent oxonol dyes exhibiting absorption and emission in the near infrared region, and methods of use thereof.

### BACKGROUND

Currently, there are a limited number of near infrared fluorophores approved for bioimaging in humans.

### BRIEF DESCRIPTION OF THE DISCLOSURE

Among the various aspects of the present disclosure is the provision of novel oxonol dye compounds for imaging compositions and methods of use thereof.

In one aspect of the present disclosure, an imaging agent composition is provided. The composition comprises at least one compound according to

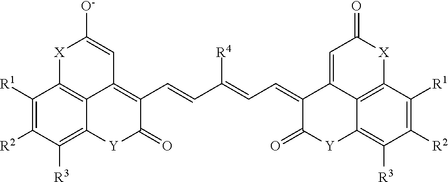

Formula (I) or resonance structure, tautomer, protonated version, or pharmaceutically acceptable salt thereof; wherein X and Y are independently selected from O and N—R⁵; and wherein R¹, R², R³, R⁴, and R⁵ are independently selected from H, alkyl, alkenyl, alkynyl, aryl, acyl, carboxy, carbalkoxy, carbamoyl, halogen, hydroxy, alkoxy, acyloxy, sulfonyloxy, alkylamino, dialkylamino, acylamino, sulfonylamino, sulfhydryl, and alkylthio.

In some embodiments, X is O and Y is O; X is O and Y is N—R⁵; X is N—R⁵ and Y is O; or X is N—R⁵ and Y is N—R⁵.

In another aspect of the present disclosure, a method of imaging a subject is provided. The method comprises: administering to the subject an imaging agent composition comprising a compound according to

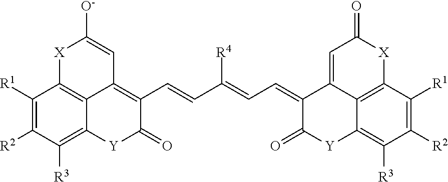

Formula (I) or resonance structure, tautomer, protonated version, or pharmaceutically acceptable salt thereof; wherein X and Y are independently selected from O and N—R⁵; wherein R¹, R², R³, R⁴, and R⁵ are independently selected from H, alkyl, alkenyl, alkynyl, aryl, acyl, carboxy, carbalkoxy, carbamoyl, halogen, hydroxy, alkoxy, acyloxy, sulfonyloxy, alkylamino, dialkylamino, acylamino, sulfonylamino, sulfhydryl, and alkylthio. The method further comprises applying an imaging modality to the subject.

In some embodiments, X is O and Y is O; X is O and Y is N—R⁵; X is N—R⁵ and Y is O; or X is N—R⁵ and Y is N—R⁵. In some embodiments, the imaging modality is a fluorescence-based optical imaging modality, such as selected from single photon imaging, multiphoton imaging, fluorescence lifetime imaging (FLIM), and Foster Resonance Energy Transfer (FRET).

In a further aspect of the present disclosure, an imaging agent precursor composition is provided. The composition comprises a 1,6-diheterophenalene-2,5-dione according to

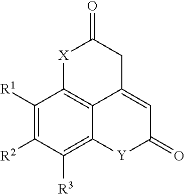

Formula (II) or resonance structure, tautomer, protonated version, or pharmaceutically acceptable salt thereof; wherein X and Y are independently selected from O and N—R⁵; and wherein R¹, R², R³, and R⁵ are independently selected from H, alkyl, alkenyl, alkynyl, aryl, acyl, carboxy, carbalkoxy, carbamoyl, halogen, hydroxy, alkoxy, acyloxy, sulfonyloxy, alkylamino, dialkylamino, acylamino, sulfonylamino, sulfhydryl, and alkylthio.

In some embodiments, X is O and Y is O; X is O, and Y is N—R⁵; X is N—R⁵, and Y is O; or X is N—R⁵ and Y is N—R⁵. In some embodiments, R⁵ is H. In some embodiments, the 1,6-diheterophenalene-2,5-dione according to Formula (II) is selected from a coumarin-derived lactone and a coumarin-derived lactam. In other embodiments, the 1,6-diheterophenalene-2,5-dione according to Formula (II) is selected from a carbostyril-derived lactone and a carbostyril-derived lactam.

In an additional aspect of the present disclosure, a method of synthesizing a compound according to

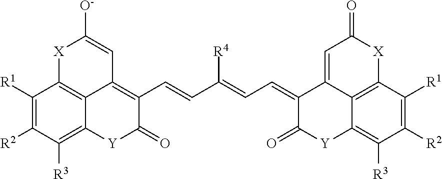

Formula (I) or resonance structure, tautomer, protonated version, or pharmaceutically acceptable salt thereof is provided. The method comprises: reacting an imaging agent precursor comprising a 1,6-diheterophenalene-2,5-dione according to

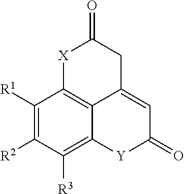

Formula (II) or resonance structure, tautomer, protonated version, or pharmaceutically acceptable salt thereof, with one of:
a Zincke salt according to

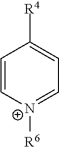

Formula (III),
a pyridine derivative according to

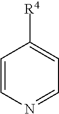

Formula (IV) in combination with an activating agent, or
a glutaconic dialdehyde according to

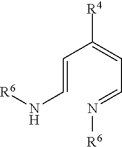

Formula (V), to produce the compound of Formula (I); wherein X and Y are independently selected from O and N—R⁵; wherein R¹, R², R³, R⁴, and R⁵ are independently selected from H, alkyl, alkenyl, alkynyl, aryl, acyl, carboxy, carbalkoxy, carbamoyl, halogen, hydroxy, alkoxy, acyloxy, sulfonyloxy, alkylamino, dialkylamino, acylamino, sulfonylamino, sulfhydryl, and alkylthio; and wherein R⁶ is independently selected from aryl, heteroaryl, sulfonyl, and acyl.

In some embodiments, the activating agent is selected from anhydrides, sulfonyl, sulfonic anhydrides, halides, acyl halides, and sulfur trioxide. In some embodiments, the glutaconic dialdehyde according to Formula (V) is selected from diimines and protonated derivatives thereof. In some embodiments, the 1,6-diheterophenalene-2,5-dione according to Formula (II) is selected from a coumarin-derived lactone, a coumarin-derived lactam, a carbostyril-derived lactone, and a carbostyril-derived lactam.

In yet another aspect of the present disclosure, a method of synthesizing a compound according to

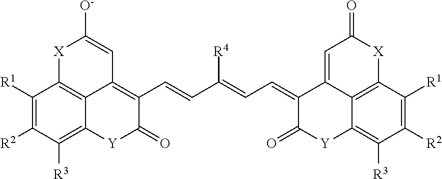

Formula (I) or resonance structure, tautomer, protonated version, or pharmaceutically acceptable salt thereof is provided. The method comprises: reacting an imaging agent precursor comprising a coumarin- or carbostyril-4-acetic acid according to

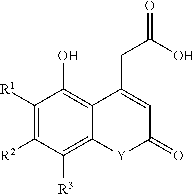

### Formula (VI)

with a pyridine derivative according to

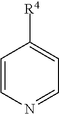

Formula (IV) in combination with an activating agent to produce the compound of Formula (I); wherein X and Y are independently selected from O and N—R⁵; and wherein R¹, R², R³, R⁴, and R⁵ are independently selected from H, alkyl, alkenyl, alkynyl, aryl, acyl, carboxy, carbalkoxy, carbamoyl, halogen, hydroxy, alkoxy, acyloxy, sulfonyloxy, alkylamino, dialkylamino, acylamino, sulfonylamino, sulfhydryl, and alkylthio.

In some embodiments, the activating agent is selected from anhydrides, sulfonyl, sulfonic anhydrides, halides, acyl halides, and sulfur trioxide.

In some embodiments, the method further comprises reacting the imaging agent precursor with the pyridine derivative in combination with the activating agent in the presence of methanesulfonyl chloride.

Other objects and features will be in part apparent and in part pointed out hereinafter.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings described herein are for illustrative purposes only. The drawings are not intended to limit the scope of the present teachings in any way.

FIG. 1 shows known carbocyanine and oxonol polymethine NIRF dyes.

FIG. 2(A-B) is an exemplary embodiment of absorption and emission spectra of dyes 4 (FIG. 2A) and 15 (FIG. 2B) in DMSO, in accordance with the present disclosure.

FIG. 3(A-B) is an exemplary embodiment of absorption and emission spectra of dyes 4 (FIG. 3A) and 15 (FIG. 3B) in methanol, in accordance with the present disclosure.

FIG. 4(A-B) is an exemplary embodiment of absorption Spectra of 4 (FIG. 4A) and 15 (FIG. 4B) in DMSO, water and methanol, in accordance with the present disclosure.

FIG. 5(A-D) is an exemplary embodiment of fluorescence intensity imaging of SKBR3-GFP-Luc cells incubated with dye 4 (FIG. 5A and FIG. 5B) and dye 15 (FIG. 5C and FIG. 5D), in accordance with the present disclosure. Incubation for 4 h at a 1 μM concentration (FIG. 5A and FIG. 5C) and for 24 h at 5 μM (FIG. 5B and FIG. 5D). NIR emission (red) was collected in the range of 760-855 nm (λ ex=715-752 nm). Scale bar: 50 μm (20×).

FIG. 6 is an exemplary embodiment of a representative FLIM image of SKBR3-GFP-Luc cells incubated with dye 15 (5 μM, 24 h), in accordance with the present disclosure. Observed cellular FLT 0.71 ns (90% contribution) and 1.36 ns (10%) from 2-exponential decay curve fitting of this image (χ²=1.06).

### DETAILED DESCRIPTION

The present disclosure is based, at least in part, on the discovery of a new class of fluorescent oxonol dyes that exhibit absorption and emission in the near infrared region. Accordingly, disclosed herein is a new class of negatively-charged oxonol NIRFs with exceptional brightness and imaging capabilities. These properties make them useful for many applications including bioimaging. As there are a limited number of near infrared fluorophores currently approved for bioimaging in humans, this new class of dyes with favorable properties enables new applications as well as commercialization in medicine and fundamental research.

In accordance with an embodiment of the present disclosure, a composition is provided. The composition includes an imaging agent according to

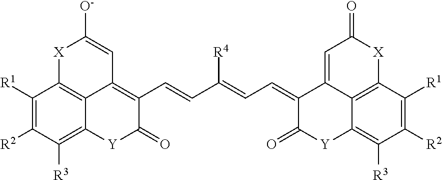

Formula (I) and resonance structures, tautomers, protonated versions, and pharmaceutically acceptable salts thereof. In some embodiments, the imaging agent absorbs and emits near infrared or infrared light.

In accordance with another embodiment, a method of imaging a subject is provided. The method includes administering to the subject a composition comprising an imaging agent according to

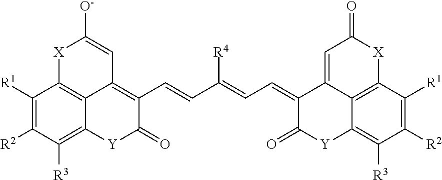

Formula (I) and resonance structures, tautomers, protonated versions, and pharmaceutically acceptable salts thereof. In some embodiments, the imaging agent absorbs and emits near infrared or infrared light. The method further includes applying an imaging modality to the subject. In some embodiments, the imaging modality is a fluorescence-based optical imaging modality, including but not limited to: single photon imaging, multiphoton imaging, fluorescence lifetime imaging (FLIM), and Foster Resonance Energy Transfer (FRET).

In accordance with a further embodiment, an imaging agent precursor composition is provided. The composition includes a 1,6-diheterophenalene-2,5-dione according to

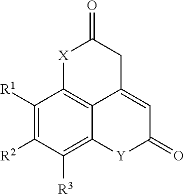

Formula (II) or resonance structure, tautomer, protonated version, or pharmaceutically acceptable salt thereof; wherein X and Y are independently selected from O and N—R⁵; and wherein R¹, R², R³, and R⁵ are independently selected from H, alkyl, alkenyl, alkynyl, aryl, acyl, carboxy, carbalkoxy, carbamoyl, halogen, hydroxy, alkoxy, acyloxy, sulfonyloxy, alkylamino, dialkylamino, acylamino, sulfonylamino, sulfhydryl, and alkylthio. In some embodiments, X is O and Y is O; X is O, and Y is N—R⁵; X is N—R⁵, and Y is O; or X is N—R⁵ and Y is N—R⁵. In some embodiments, R⁵ is H. In some embodiments, the 1,6-diheterophenalene-2,5-dione according to Formula (II) is selected from a coumarin-derived lactone and a coumarin-derived lactam. In other embodiments, the 1,6-diheterophenalene-2,5-dione according to Formula (II) is selected from a carbostyril-derived lactone and a carbostyril-derived lactam.

In some embodiments, the oxonol imaging agent compounds, precursors, and/or compositions thereof absorb and emit light in the near infrared region and are negatively charged.

In accordance with an additional embodiment, a method of synthesizing a compound according to

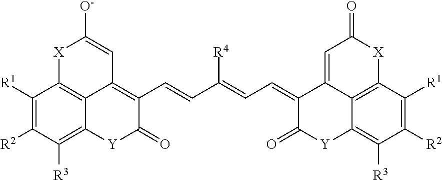

Formula (I) or resonance structure, tautomer, protonated version, or pharmaceutically acceptable salt thereof is provided. The method includes: reacting an imaging agent precursor comprising a 1,6-diheterophenalene-2,5-dione according to

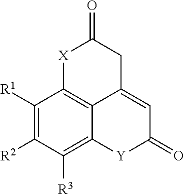

Formula (II) or resonance structure, tautomer, protonated version, or pharmaceutically acceptable salt thereof, with one of:
a Zincke salt according to

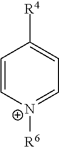

Formula (III),
a pyridine derivative according to

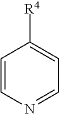

Formula (IV) in combination with an activating agent, or
a glutaconic dialdehyde according to

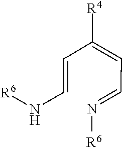

Formula (V), to produce the compound of Formula (I); wherein X and Y are independently selected from O and N—R⁵; wherein R¹, R², R³, R⁴, and R⁵ are independently selected from H, alkyl, alkenyl, alkynyl, aryl, acyl, carboxy, carbalkoxy, carbamoyl, halogen, hydroxy, alkoxy, acyloxy, sulfonyloxy, alkylamino, dialkylamino, acylamino, sulfonylamino, sulfhydryl, and alkylthio; and wherein R⁶ is independently selected from aryl, heteroaryl, sulfonyl, and acyl.

In some embodiments, the activating agent is selected from anhydrides, sulfonyl, sulfonic anhydrides, halides, acyl halides, and sulfur trioxide. In some embodiments, the glutaconic dialdehyde according to Formula (V) is selected from diimines and protonated derivatives thereof. In some embodiments, the 1,6-diheterophenalene-2,5-dione according to Formula (II) is selected from a coumarin-derived lactone, a coumarin-derived lactam, a carbostyril-derived lactone, and a carbostyril-derived lactam.

In accordance with yet another embodiment, a method of synthesizing a compound according to

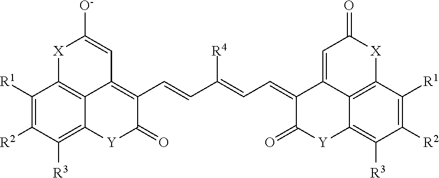

Formula (I) or resonance structure, tautomer, protonated version, or pharmaceutically acceptable salt thereof is provided. The method includes: reacting an imaging agent precursor comprising a coumarin- or carbostyril-4-acetic acid according to

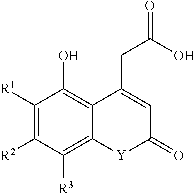

Formula (VI) with a pyridine derivative according to

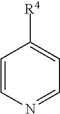

Formula (IV) in combination with an activating agent to produce the compound of Formula (I); wherein X and Y are independently selected from O and N—R⁵; and wherein R¹, R², R³, R⁴, and R⁵ are independently selected from H, alkyl, alkenyl, alkynyl, aryl, acyl, carboxy, carbalkoxy, carbamoyl, halogen, hydroxy, alkoxy, acyloxy, sulfonyloxy, alkylamino, dialkylamino, acylamino, sulfonylamino, sulfhydryl, and alkylthio. In some embodiments, the activating agent is selected from anhydrides, sulfonyl, sulfonic anhydrides, halides, acyl halides, and sulfur trioxide. In some embodiments, the method further includes reacting the imaging agent precursor with the pyridine derivative in combination with the activating agent in the presence of methanesulfonyl chloride.

### Chemical Agent

Examples of oxonol dye and imaging agent compounds and compositions, including those for use as imaging agents in bioimaging applications, are described herein. Oxonol dyes and oxonol imaging agents include compounds according to the formulas:

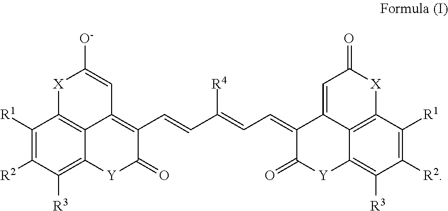

Precursors of oxonol imaging agents include:
a 1,6-diheterophenalene-2,5-dione according to

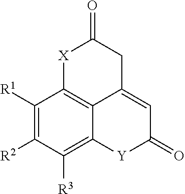

Formula (II),
a Zincke salt according to

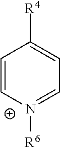

Formula (III),
a pyridine derivative according to

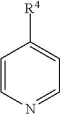

Formula (IV),
a glutaconic dialdehyde according to

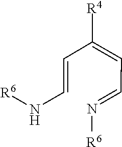

Formula (V), and
a coumarin- or carbostyril-4-acetic acid according to

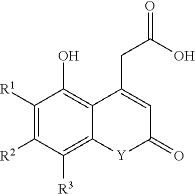

Formula (VI).

In exemplary embodiments of the present disclosure: X and Y are independently selected from O and N—R⁵; R¹, R², R³, R⁴, and R⁵ are independently selected from H, alkyl, alkenyl, alkynyl, aryl, acyl, carboxy, carbalkoxy, carbamoyl, halogen, hydroxy, alkoxy, acyloxy, sulfonyloxy, alkylamino, dialkylamino, acylamino, sulfonylamino, sulfhydryl, and alkylthio; and R⁶ is independently selected from aryl, heteroaryl, sulfonyl, and acyl.

Compounds of Formulas (II)-(VI) may be used as disclosed herein for synthesizing the compounds of Formula (I). Depending upon the embodiment, methods of synthesizing the compounds of Formula (I) may include reaction(s) performed in the presence of methanesulfonyl chloride. Activating agent include, but are not limited to anhydrides, sulfonyl, sulfonic anhydrides, halides, acyl halides, and sulfur trioxide. Glutaconic dialdehyde compounds according to Formula (V) include, but are not limited to diimines thereof and protonated derivatives thereof. 1,6-diheterophenalene-2,5-dione compounds according to Formula (II) include, but are not limited to a coumarin-derived lactone, a coumarin-derived lactam, a carbostyril-derived lactone, and a carbostyril-derived lactam.

In general, the formulas, analogs, and R groups of the present disclosure can be optionally substituted or functionalized with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; a C₂₋₁₀cycloalkyl optionally containing unsaturation or one oxygen or nitrogen atom; straight chain or branched C₁₋₁₀alkyl amine; heterocyclyl; heterocyclic amine; and aryl comprising a phenyl; heteroaryl containing from 1 to 4 N, O, or S atoms; unsubstituted phenyl ring; substituted phenyl ring; unsubstituted heterocyclyl; and substituted heterocyclyl, wherein the unsubstituted phenyl ring or substituted phenyl ring can be optionally substituted with one or more groups independently selected from the group consisting of hydroxyl; C₁₋₁₀alkyl hydroxyl; amine; C₁₋₁₀carboxyl; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂₋₁₀cycloalkyl optionally containing unsaturation or one oxygen or nitrogen atom; straight chain or branched C₁₋₁₀alkyl amine; heterocyclyl; heterocyclic amine; aryl comprising a phenyl; and heteroaryl containing from 1 to 4 N, O, or S atoms; and the unsubstituted heterocyclyl or substituted heterocyclyl can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂₋₁₀cycloalkyl optionally containing unsaturation or one oxygen or nitrogen atom; heterocyclyl; straight chain or branched C₁₋₁₀alkyl amine; heterocyclic amine; and aryl comprising a phenyl; and heteroaryl containing from 1 to 4 N, O, or S atoms. Any of the above can be further optionally substituted.

The term “imine” or “imino”, as used herein, unless otherwise indicated, can include a functional group or chemical compound containing a carbon-nitrogen double bond. The expression “imino compound”, as used herein, unless otherwise indicated, refers to a compound that includes an “imine” or an “imino” group as defined herein. The “imine” or “imino” group can be optionally substituted.

The term “hydroxyl”, as used herein, unless otherwise indicated, can include —OH. The “hydroxyl” can be optionally substituted.

The terms “halogen” and “halo”, as used herein, unless otherwise indicated, include a chlorine, chloro, Cl; fluorine, fluoro, F; bromine, bromo, Br; or iodine, iodo, or I.

The term “acetamide”, as used herein, is an organic compound with the formula CH₃CONH₂. The “acetamide” can be optionally substituted.

The term “aryl”, as used herein, unless otherwise indicated, include a carbocyclic aromatic group. Examples of aryl groups include, but are not limited to, phenyl, benzyl, naphthyl, or anthracenyl. The “aryl” can be optionally substituted.

The terms “amine” and “amino”, as used herein, unless otherwise indicated, include a functional group that contains a nitrogen atom with a lone pair of electrons and wherein one or more hydrogen atoms have been replaced by a substituent such as, but not limited to, an alkyl group or an aryl group. The “amine” or “amino” group can be optionally substituted.

The term “alkyl”, as used herein, unless otherwise indicated, can include saturated monovalent hydrocarbon radicals having straight or branched moieties, such as but not limited to, methyl, ethyl, propyl, butyl, pentyl, hexyl, octyl groups, etc. Representative straight-chain lower alkyl groups include, but are not limited to, -methyl, -ethyl, -n-propyl, -n-butyl, -n-pentyl, -n-hexyl, -n-heptyl and -n-octyl; while branched lower alkyl groups include, but are not limited to, -isopropyl, -sec-butyl, -isobutyl, -tert-butyl, -isopentyl, 2-methylbutyl, 2-methylpentyl, 3-methylpentyl, 2,2-dimethylbutyl, 2,3-dimethylbutyl, 2,2-dimethylpentyl, 2,3-dimethylpentyl, 3,3-dimethylpentyl, 2,3,4-trimethylpentyl, 3-methylhexyl, 2,2-dimethylhexyl, 2,4-dimethylhexyl, 2,5-dimethylhexyl, 3,5-dimethylhexyl, 2,4-dimethylpentyl, 2-methylheptyl, 3-methylheptyl, unsaturated C₁₋₁₀ alkyls include, but are not limited to, -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, 1-hexyl, 2-hexyl, 3-hexyl, -acetylenyl, -propynyl, -1-butynyl, -2-butynyl, -1-pentynyl, -2-pentynyl, or -3-methyl-1 butynyl. An alkyl can be saturated, partially saturated, or unsaturated. The “alkyl” can be optionally substituted.

The term “carboxyl”, as used herein, unless otherwise indicated, can include a functional group consisting of a carbon atom double bonded to an oxygen atom and single bonded to a hydroxyl group (—COOH). The “carboxyl” can be optionally substituted.

The term “carbonyl”, as used herein, unless otherwise indicated, can include a functional group consisting of a carbon atom double-bonded to an oxygen atom (C=O). The “carbonyl” can be optionally substituted.

The term “alkenyl”, as used herein, unless otherwise indicated, can include alkyl moieties having at least one carbon-carbon double bond wherein alkyl is as defined above and including E and Z isomers of said alkenyl moiety. An alkenyl can be partially saturated or unsaturated. The “alkenyl” can be optionally substituted.

The term “alkynyl”, as used herein, unless otherwise indicated, can include alkyl moieties having at least one carbon-carbon triple bond wherein alkyl is as defined above. An alkynyl can be partially saturated or unsaturated. The “alkynyl” can be optionally substituted.

The term “acyl”, as used herein, unless otherwise indicated, can include a functional group derived from an aliphatic carboxylic acid, by removal of the hydroxyl (—OH) group. The “acyl” can be optionally substituted.

The term “alkoxyl”, as used herein, unless otherwise indicated, can include O-alkyl groups wherein alkyl is as defined above and O represents oxygen. Representative alkoxyl groups include, but are not limited to, —O-methyl, —O-ethyl, —O-n-propyl, —O-n-butyl, —O-n-pentyl, —O-n-hexyl, —O-n-heptyl, —O-n-octyl, —O-isopropyl, —O-sec-butyl, —O-isobutyl, —O-tert-butyl, —O-isopentyl, —O-2-methylbutyl, —O-2-methylpentyl, —O-3-methylpentyl, —O-2,2-dimethylbutyl, —O-2,3-dimethylbutyl, —O-2,2-dimethylpentyl, —O-2,3-dimethylpentyl, —O-3,3-dimethylpentyl, —O-2,3,4-trimethylpentyl, —O-3-methylhexyl, —O-2,2-dimethylhexyl, —O-2,4-dimethylhexyl, —O-2,5-dimethylhexyl, —O-3,5-dimethylhexyl, —O-2,4dimethylpentyl, —O-2-methylheptyl, —O-3-methylheptyl, —O-vinyl, —O-allyl, —O-1-butenyl, —O-2-butenyl, —O— isobutylenyl, —O-1-pentenyl, —O-2-pentenyl, —O-3-methyl-1-butenyl, —O-2-methyl-2-butenyl, —O-2,3-dimethyl-2-butenyl, —O-1-hexyl, —O-2-hexyl, —O-3-hexyl, —O-acetylenyl, —O— propynyl, —O-1-butynyl, —O-2-butynyl, —O-1-pentynyl, —O-2-pentynyl and —O-3-methyl-1-butynyl, —O-cyclopropyl, —O-cyclobutyl, —O-cyclopentyl, —O-cyclohexyl, —O-cycloheptyl, —O— cyclooctyl, —O-cyclononyl and —O-cyclodecyl, —O—CH₂-cyclopropyl, —O—CH₂-cyclobutyl, —O—CH₂-cyclopentyl, —O—CH₂-cyclohexyl, —O—CH₂-cycloheptyl, —O—CH₂-cyclooctyl, —O—CH₂-cyclononyl, —O—CH₂-cyclodecyl, —O—(CH₂)₂-cyclopropyl, —O—(CH₂)₂-cyclobutyl, —O—(CH₂)₂-cyclopentyl, —O—(CH₂)₂-cyclohexyl, —O—(CH₂)₂-cycloheptyl, —O—(CH₂)₂-cyclooctyl, —O—(CH₂)₂-cyclononyl, or —O—(CH₂)₂-cyclodecyl. An alkoxyl can be saturated, partially saturated, or unsaturated. The “alkoxyl” can be optionally substituted.

The term “cycloalkyl”, as used herein, unless otherwise indicated, can include an aromatic, a non-aromatic, saturated, partially saturated, or unsaturated, monocyclic or fused, spiro or unfused bicyclic or tricyclic hydrocarbon referred to herein containing a total of from 1 to 10 carbon atoms (e.g., 1 or 2 carbon atoms if there are other heteroatoms in the ring), preferably 3 to 8 ring carbon atoms. Examples of cycloalkyls include, but are not limited to, C₃₋₁₀ cycloalkyl groups include, but are not limited to, -cyclopropyl, -cyclobutyl, -cyclopentyl, -cyclopentadienyl, -cyclohexyl, -cyclohexenyl, -1,3-cyclohexadienyl, -1,4-cyclohexadienyl, -cycloheptyl, -1,3-cycloheptadienyl, -1,3,5-cycloheptatrienyl, -cyclooctyl, and -cyclooctadienyl. The term “cycloalkyl” also can include -lower alkyl-cycloalkyl, wherein lower alkyl and cycloalkyl are as defined herein. Examples of -lower alkyl-cycloalkyl groups include, but are not limited to, —CH₂-cyclopropyl, —CH₂-cyclobutyl, —CH₂-cyclopentyl, —CH₂-cyclopentadienyl, —CH₂-cyclohexyl, —CH₂-cycloheptyl, or —CH₂-cyclooctyl. The “cycloalkyl” can be optionally substituted. A “cycloheteroalkyl”, as used herein, unless otherwise indicated, can include any of the above with a carbon substituted with a heteroatom (e.g., O, S, N).

The term “heterocyclic” or “heteroaryl”, as used herein, unless otherwise indicated, can include an aromatic or non-aromatic cycloalkyl in which one to four of the ring carbon atoms are independently replaced with a heteroatom from the group consisting of O, S, and N. Representative examples of a heterocycle include, but are not limited to, benzofuranyl, benzothiophene, indolyl, benzopyrazolyl, coumarinyl, isoquinolinyl, pyrrolyl, pyrrolidinyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl, (1,4)-dioxane, (1,3)-dioxolane, 4,5-dihydro-1H-imidazolyl, or tetrazolyl. Heterocycles can be substituted or unsubstituted. Heterocycles can also be bonded at any ring atom (i.e., at any carbon atom or heteroatom of the heterocyclic ring). A heterocyclic can be saturated, partially saturated, or unsaturated. The “heterocyclic” can be optionally substituted.

The term “indole”, as used herein, is an aromatic heterocyclic organic compound with formula C₈H₇N. It has a bicyclic structure, consisting of a six-membered benzene ring fused to a five-membered nitrogen-containing pyrrole ring. The “indole” can be optionally substituted.

The term “cyano”, as used herein, unless otherwise indicated, can include a —CN group. The “cyano” can be optionally substituted.

The term “alcohol”, as used herein, unless otherwise indicated, can include a compound in which the hydroxyl functional group (—OH) is bound to a carbon atom. In particular, this carbon center should be saturated, having single bonds to three other atoms. The “alcohol” can be optionally substituted.

The term “solvate” is intended to mean a solvate form of a specified compound that retains the effectiveness of such compound. Examples of solvates include compounds of the invention in combination with, for example, water, isopropanol, ethanol, methanol, dimethylsulfoxide (DMSO), ethyl acetate, acetic acid, or ethanolamine.

The term “mmol”, as used herein, is intended to mean millimole. The term “equiv”, as used herein, is intended to mean equivalent. The term “mL”, as used herein, is intended to mean milliliter. The term “g”, as used herein, is intended to mean gram. The term “kg”, as used herein, is intended to mean kilogram. The term “μg”, as used herein, is intended to mean micrograms. The term “h”, as used herein, is intended to mean hour. The term “min”, as used herein, is intended to mean minute. The term “M”, as used herein, is intended to mean molar. The term “μL”, as used herein, is intended to mean microliter. The term “μM”, as used herein, is intended to mean micromolar. The term “nM”, as used herein, is intended to mean nanomolar. The term “N”, as used herein, is intended to mean normal. The term “amu”, as used herein, is intended to mean atomic mass unit. The term “° C.”, as used herein, is intended to mean degree Celsius. The term “wt/wt”, as used herein, is intended to mean weight/weight. The term “v/v”, as used herein, is intended to mean volume/volume. The term “MS”, as used herein, is intended to mean mass spectroscopy. The term “HPLC”, as used herein, is intended to mean high performance liquid chromatograph. The term “RT”, as used herein, is intended to mean room temperature. The term “e.g.,”, as used herein, is intended to mean example. The term “N/A”, as used herein, is intended to mean not tested.

As used herein, the expression “pharmaceutically acceptable salt” refers to pharmaceutically acceptable organic or inorganic salts of a compound of the invention. Preferred salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, or pamoate (i.e., 1,1′-methylene-bis-(2-hydroxy-3-naphthoate)) salts. A pharmaceutically acceptable salt may involve the inclusion of another molecule such as an acetate ion, a succinate ion, or another counterion. The counterion may be any organic or inorganic moiety that stabilizes the charge on the parent compound. Furthermore, a pharmaceutically acceptable salt may have more than one charged atom in its structure. In instances where multiple charged atoms are part of the pharmaceutically acceptable salt, the pharmaceutically acceptable salt can have multiple counterions. Hence, a pharmaceutically acceptable salt can have one or more charged atoms and/or one or more counterion. As used herein, the expression “pharmaceutically acceptable solvate” refers to an association of one or more solvent molecules and a compound of the invention. Examples of solvents that form pharmaceutically acceptable solvates include, but are not limited to, water, isopropanol, ethanol, methanol, DMSO, ethyl acetate, acetic acid, and ethanolamine. As used herein, the expression “pharmaceutically acceptable hydrate” refers to a compound of the invention, or a salt thereof, that further can include a stoichiometric or non-stoichiometric amount of water bound by non-covalent intermolecular forces.

### Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The term “transfection,” as used herein, refers to the process of introducing nucleic acids into cells by non-viral methods. The term “transduction,” as used herein, refers to the process whereby foreign DNA is introduced into another cell via a viral vector.

The terms “heterologous DNA sequence”, “exogenous DNA segment”, or “heterologous nucleic acid”, “transgene”, “exogenous polynucleotide” as used herein, each refers to a sequence that originates from a source foreign (e.g., non-native) to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Sequences described herein can also be the reverse, the complement, or the reverse complement of the nucleotide sequences described herein. The RNA goes in the reverse direction compared to the DNA, but its base pairs still match (e.g., G to C). The reverse complementary RNA for a positive strand DNA sequence will be identical to the corresponding negative strand DNA sequence. Reverse complement converts a DNA sequence into its reverse, complement, or reverse-complement counterpart.

| Base | Name | Bases Represented | Complementary Base |
|---|---|---|---|
| A | Adenine | A | T |
| T | Thymidine | T | A |
| U | Uridine(RNA only) | U | A |
| G | Guanidine | G | C |
| C | Cytidine | C | G |
| Y | pYrimidine | C T | R |
| R | puRine | A G | Y |
| S | Strong(3Hbonds) | G C | S* |
| W | Weak(2Hbonds) | A T | W* |
| K | Keto | T/U G | M |
| M | aMino | A C | K |
| B | not A | C G T | V |
| D | not C | A G T | H |
| H | not G | A C T | D |
| V | not T/U | A C G | B |
| N | Unknown | A C G T | N |

Complementarity is a property shared between two nucleic acid sequences (e.g., RNA, DNA), such that when they are aligned antiparallel to each other, the nucleotide bases at each position will be complementary. Two bases are complementary if they form Watson-Crick base pairs.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

An “expression vector”, otherwise known as an “expression construct”, is generally a plasmid or virus designed for gene expression in cells. The vector is used to introduce a specific gene into a target cell, and can commandeer the cell's mechanism for protein synthesis to produce the protein encoded by the gene. Expression vectors are the basic tools in biotechnology for the production of proteins. The vector is engineered to contain regulatory sequences that act as enhancer and/or promoter regions and lead to efficient transcription of the gene carried on the expression vector. The goal of a well-designed expression vector is the efficient production of protein, and this may be achieved by the production of significant amount of stable messenger RNA, which can then be translated into protein. The expression of a protein may be tightly controlled, and the protein is only produced in significant quantity when necessary through the use of an inducer, in some systems however the protein may be expressed constitutively. As described herein, Escherichia coli is used as the host for protein production, but other cell types may also be used.

In molecular biology, an “inducer” is a molecule that regulates gene expression. An inducer can function in two ways, such as:

(i) By disabling repressors. The gene is expressed because an inducer binds to the repressor. The binding of the inducer to the repressor prevents the repressor from binding to the operator. RNA polymerase can then begin to transcribe operon genes. An operon is a cluster of genes that are transcribed together to give a single messenger RNA (mRNA) molecule, which therefore encodes multiple proteins.

(ii) By binding to activators. Activators generally bind poorly to activator DNA sequences unless an inducer is present. An activator binds to an inducer and the complex binds to the activation sequence and activates target gene. Removing the inducer stops transcription. Because a small inducer molecule is required, the increased expression of the target gene is called induction.

Repressor proteins bind to the DNA strand and prevent RNA polymerase from being able to attach to the DNA and synthesize mRNA. Inducers bind to repressors, causing them to change shape and preventing them from binding to DNA. Therefore, they allow transcription, and thus gene expression, to take place.

For a gene to be expressed, its DNA sequence (or polynucleotide sequence) must be copied (in a process known as transcription) to make a smaller, mobile molecule called messenger RNA (mRNA), which carries the instructions for making a protein to the site where the protein is manufactured (in a process known as translation). Many different types of proteins can affect the level of gene expression by promoting or preventing transcription. In prokaryotes (such as bacteria), these proteins often act on a portion of DNA known as the operator at the beginning of the gene. The promoter is where RNA polymerase, the enzyme that copies the genetic sequence and synthesizes the mRNA, attaches to the DNA strand.

Some genes are modulated by activators, which have the opposite effect on gene expression as repressors. Inducers can also bind to activator proteins, allowing them to bind to the operator DNA where they promote RNA transcription. Ligands that bind to deactivate activator proteins are not, in the technical sense, classified as inducers, since they have the effect of preventing transcription.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “ribosome binding site”, or “ribosomal binding site (RBS)”, refers to a sequence of nucleotides upstream of the start codon of an mRNA transcript that is responsible for the recruitment of a ribosome during the initiation of translation. Generally, RBS refers to bacterial sequences, although internal ribosome entry sites (IRES) have been described in mRNAs of eukaryotic cells or viruses that infect eukaryotes. Ribosome recruitment in eukaryotes is generally mediated by the 5′ cap present on eukaryotic mRNAs.

A ribosomal skipping sequence (e.g., 2A sequence such as furin-GSG-T2A) can be used in a construct to prevent covalently linking translated amino acid sequences.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using self-replicating primers, paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above-required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A. For example, the percent identity can be at least 80% or about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

Substitution refers to the replacement of one amino acid with another amino acid in a protein or the replacement of one nucleotide with another in DNA or RNA. Insertion refers to the insertion of one or more amino acids in a protein or the insertion of one or more nucleotides with another in DNA or RNA. Deletion refers to the deletion of one or more amino acids in a protein or the deletion of one or more nucleotides with another in DNA or RNA. Generally, substitutions, insertions, or deletions can be made at any position so long as the required activity is retained.

“Point mutation” refers to when a single base pair is altered. A point mutation or substitution is a genetic mutation where a single nucleotide base is changed, inserted, or deleted from a DNA or RNA sequence of an organism's genome. Point mutations have a variety of effects on the downstream protein product-consequences that are moderately predictable based upon the specifics of the mutation. These consequences can range from no effect (e.g., synonymous mutations) to deleterious effects (e.g., frameshift mutations), with regard to protein production, composition, and function. Point mutations can have one of three effects. First, the base substitution can be a silent mutation where the altered codon corresponds to the same amino acid. Second, the base substitution can be a missense mutation where the altered codon corresponds to a different amino acid. Or third, the base substitution can be a nonsense mutation where the altered codon corresponds to a stop signal. Silent mutations result in a new codon (a triplet nucleotide sequence in RNA) that codes for the same amino acid as the wild type codon in that position. In some silent mutations the codon codes for a different amino acid that happens to have the same properties as the amino acid produced by the wild type codon. Missense mutations involve substitutions that result in functionally different amino acids; these can lead to alteration or loss of protein function. Nonsense mutations, which are a severe type of base substitution, result in a stop codon in a position where there was not one before, which causes the premature termination of protein synthesis and can result in a complete loss of function in the finished protein.

Generally, conservative substitutions can be made at any position so long as the required activity is retained. So-called conservative exchanges can be carried out in which the amino acid which is replaced has a similar property as the original amino acid, for example, the exchange of Glu by Asp, Gln by Asn, Val by lie, Leu by lie, and Ser by Thr. For example, amino acids with similar properties can be Aliphatic amino acids (e.g., Glycine, Alanine, Valine, Leucine, Isoleucine); hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na⁺])+0.41 (fraction G/C content)−0.63 (% formamide)−(600/l). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transformed cells can be selected and propagated to provide recombinant host cells that comprise the expression vector stably integrated in the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| Side Chain Characteristic | Amino Acid |
|---|---|
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | DE |

Conservative Substitutions III

| | Exemplary |
|---|---|
| Original Residue | Substitution |
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| | Leu, Val, Met, Ala, |
| Ile (I) | Phe, |
| Leu (L) | Ile, Val, Met, Ala, Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, Ala |

Exemplary nucleic acids that may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), single guide RNA (sgRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

### Formulation

The agents and compositions described herein can be formulated by any conventional manner using one or more pharmaceutically acceptable carriers or excipients as described in, for example, Remington's Pharmaceutical Sciences (A.R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc., may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A.R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently, affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

### Therapeutic Methods

Also provided is a process of bioimaging of a subject by administering an effective amount of an oxonol dye imaging agent, and applying an imaging modality to the subject.

Methods described herein are generally performed on a subject in need of imaging. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of an oxonol imaging agent is, for example, an amount that would cause the desired therapeutic effect in a subject while minimizing undesired side effects. In various embodiments, an effective amount of oxonol imaging agent described herein can substantially dye a target area of the subject with minor to no side effects, while maintaining imaging potency for a desired length of time needed to obtain an image (or images) based on the imaging modality utilized. Imaging modalities are those known in the art capable of detecting, imaging, quantifying, and/or recording fluorescence signals/emissions from the imaging agents/dyes disclosed herein.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of oxonol imaging agent can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to successfully image the subject.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; the activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes reversing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or a physician.

Administration of an oxonol imaging agent can occur as a single event or over a time course of treatment. For example, an oxonol imaging agent can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to or before, concurrent with, or after conventional treatment modalities for imaging.

An oxonol imaging agent can be administered simultaneously or sequentially with another imaging agent, an antibiotic, an anti-inflammatory, or another agent. For example, an oxonol imaging agent can be administered simultaneously with another agent, such as an antibiotic or an anti-inflammatory. Simultaneous administration can occur through administration of separate compositions, each containing one or more of an oxonol imaging agent, an antibiotic, an anti-inflammatory, or another agent. Simultaneous administration can occur through administration of one composition containing two or more of an oxonol imaging agent, an antibiotic, an anti-inflammatory, or another agent. An oxonol imaging agent can be administered sequentially with a different imaging agent, an antibiotic, an anti-inflammatory, or another agent. For example, an oxonol imaging agent can be administered before or after administration of an antibiotic, an anti-inflammatory, or another agent.

Active compounds are administered at a therapeutically effective dosage sufficient to treat a condition associated with a condition in a patient. For example, the efficacy of a compound can be evaluated in an animal model system that may be predictive of efficacy in treating the disease in a human or another animal, such as the model systems shown in the examples and drawings.

An effective dose range of a therapeutic can be extrapolated from effective doses determined in animal studies for a variety of different animals. In general, a human equivalent dose (HED) in mg/kg can be calculated in accordance with the following formula (see e.g., Reagan-Shaw et al., FASEB J., 22(3):659-661, 2008, which is incorporated herein by reference):

HED (mg/kg)=Animal dose (mg/kg)×(Animal Kₘ/Human Kₘ)

Use of the Kₘ factors in conversion results in more accurate HED values, which are based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are peculiar to each individual. Nonetheless, a calculated HED dose provides a general guide. Other factors affecting the dose include the physical and clinical state of the patient, the route of administration, the intended goal of treatment, and the potency, stability, and toxicity of the particular therapeutic formulation.

The actual dosage amount of a compound of the present disclosure or composition comprising a compound of the present disclosure administered to a subject may be determined by physical and physiological factors such as type of animal treated, age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

In some embodiments, the oxonol imaging agent may be administered in an amount from about 1 mg/kg to about 100 mg/kg, or about 1 mg/kg to about 50 mg/kg, or about 1 mg/kg to about 25 mg/kg, or about 1 mg/kg to about 15 mg/kg, or about 1 mg/kg to about 10 mg/kg, or about 1 mg/kg to about 5 mg/kg, or about 3 mg/kg. In some embodiments, an oxonol imaging agent such as a compound described herein, e.g., of formulas (I)-(VI) may be administered in a range of about 1 mg/kg to about 200 mg/kg, or about 50 mg/kg to about 200 mg/kg, or about 50 mg/kg to about 100 mg/kg, or about 75 mg/kg to about 100 mg/kg, or about 100 mg/kg.

The effective amount may be less than 1 mg/kg/day, less than 500 mg/kg/day, less than 250 mg/kg/day, less than 100 mg/kg/day, less than 50 mg/kg/day, less than 25 mg/kg/day or less than 10 mg/kg/day. It may alternatively be in the range of 1 mg/kg/day to 200 mg/kg/day.

In other non-limiting examples, a dose may also comprise from about 1 micro-gram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

### Cell Therapy

Cells generated according to the methods described herein can be used in cell therapy. Cell therapy (also called cellular therapy, cell transplantation, or cytotherapy) can be a therapy in which viable cells are injected, grafted, or implanted into a patient in order to effectuate a medicinal effect or therapeutic benefit. For example, transplanting T-cells capable of fighting cancer cells via cell-mediated immunity can be used in the course of immunotherapy, grafting stem cells can be used to regenerate diseased tissues, or transplanting beta cells can be used to treat diabetes.

Stem cell and cell transplantation has gained significant interest by researchers as a potential new therapeutic strategy for a wide range of diseases, in particular for degenerative and immunogenic pathologies.

Allogeneic cell therapy or allogenic transplantation uses donor cells from a different subject than the recipient of the cells. A benefit of an allogeneic strategy is that unmatched allogenic cell therapies can form the basis of “off the shelf” products.

Autologous cell therapy or autologous transplantation uses cells that are derived from the subject's own tissues. It could also involve the isolation of matured cells from diseased tissues, to be later re-implanted at the same or neighboring tissues. A benefit of an autologous strategy is that there is limited concern for immunogenic responses or transplant rejection.

Xenogeneic cell therapies or xenotransplantation uses cells from another species. For example, pig derived cells can be transplanted into humans. Xenogeneic cell therapies can involve human cell transplantation into experimental animal models for assessment of efficacy and safety or enable xenogeneic strategies to humans as well.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency; improve taste of the product; or improve shelf life of the product.

### Screening

Also provided are screening methods.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 MW, or less than about 1000 MW, or less than about 800 MW) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example, ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character x log P of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character xlogP of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict the bioavailability of a compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

### Imaging Modalities

Imaging modalities that may be applied to a subject are broadly defined herein to include optical imaging methods that are based on fluorescence. Depending upon the embodiment, an imaging modality can be selected from single photon imaging, multiphoton imaging, fluorescence lifetime imaging (FLIM), Foster Resonance Energy Transfer (FRET). In exemplary embodiments, an imaging agent composition according to the present disclosure is administered to a subject, followed by application of an imaging modality to the subject. Imaging agents compositions according to the present disclosure comprise at least one compound (e.g., an imaging dye) according to Formula (I) as disclosed herein.

### Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to oxonol imaging agents and/or precursors thereof. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or another substrate, and/or may be supplied as an electronic-readable medium or video. Detailed instructions may not be physically associated with the kit; instead, a user may be directed to an Internet web site specified by the manufacturer or distributor of the kit.

A control sample or a reference sample as described herein can be a sample from a healthy subject or sample, a wild-type subject or sample, or from populations thereof. A reference value can be used in place of a control or reference sample, which was previously obtained from a healthy subject or a group of healthy subjects or a wild-type subject or sample. A control sample or a reference sample can also be a sample with a known amount of a detectable compound or a spiked sample.

The methods and algorithms of the invention may be enclosed in a controller or processor. Furthermore, methods and algorithms of the present invention, can be embodied as a computer-implemented method or methods for performing such computer-implemented method or methods, and can also be embodied in the form of a tangible or non-transitory computer-readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer program include, for example, floppy disks and diskettes, compact disk (CD)-ROMs (whether or not writeable), DVD digital disks, RAM and ROM memories, computer hard drives and back-up drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general-purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1

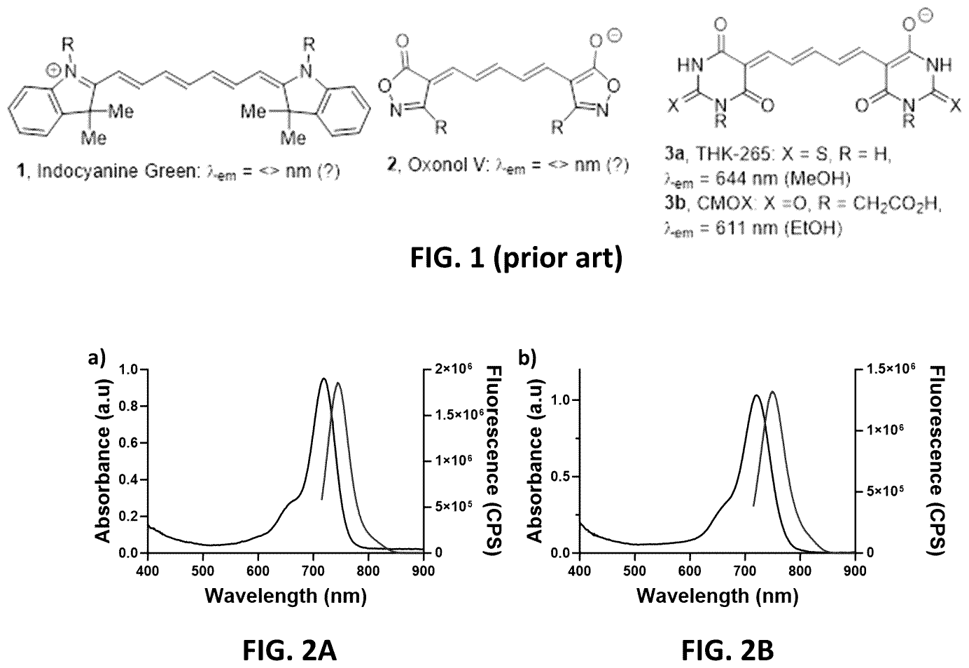

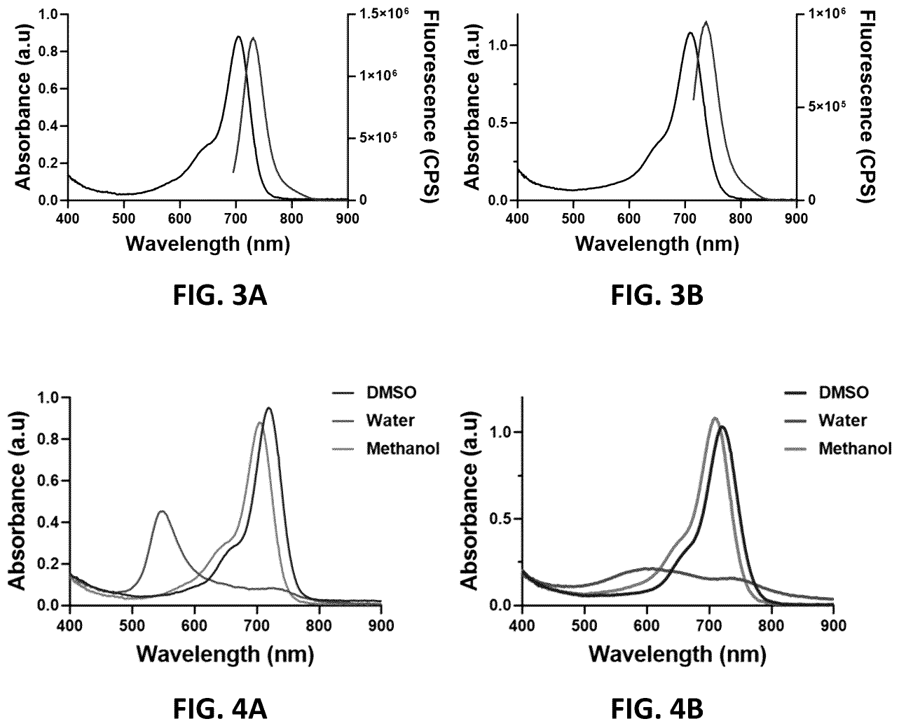

According to the present disclosure, a new class of biologically relevant near-infrared (NIR) dyes have been developed with a unique negatively charged structure of fluorescent core. While negatively charged fluorophores such as oxonols (see FIG. 1, compounds 2 and 3) carry a negative charge at physiological pH, they are optically active only in the visible and red parts of the spectrum. Negatively charged fluorophore emitters in the NIR part of the spectrum (and beyond) are not known and hence their biological properties have not been explored. Addressing this gap, a set of compounds were synthesized that are negatively charged and exhibit strong emission in the NIR part of the spectrum.

Biologically relevant near infrared fluorescent (NIRF) dyes are a class of organic molecules that absorb and emit light within the near infrared spectrum (700-900 nm and beyond), offering deeper tissue penetration and reduced autofluorescence for enhanced imaging contrast in biological tissues compared to visible light. These dyes are particularly valued in medical and biological fields for their ability to be seen through a relative thick biological tissue enabling clinical and preclinical non-invasive in vivo imaging, surgical guidance, drug delivery monitoring, and diagnostic imaging.

Most up-to-date NIR fluorophores, including the FDA-approved indocyanine green, feature a cationic carbocyanine core, such as the FDA-approved indocyanine green (FIG. 1, structure 1), feature a cationic carbocyanine core. This structural core made from a set of conjugated methine units enables strong absorption and emission in the NIR spectral range. The synthetic flexibility of these molecules enables the generation of various contrast agents that differ primarily in hydrophilicity, solubility, and specific targeting capabilities as well as functional activity (through conjugating to targeting groups, of functionally active (i.e., reactive oxygen species sensors) and many others. Such versatility allows for the tailoring of cationic cyanine fluorophores to meet the diverse requirements of biological imaging, from enhancing water solubility for improved circulation times in vivo to modifying the structure for targeted binding to specific tissues or molecules, and functional activation as a result of the enzymatic or another type of chemical reactions inside the body. The adaptability of the carbocyanine core thus plays a crucial role in the widespread application of NIR fluorophores across different domains of medical imaging and diagnostic procedures, offering a broad palette for designing functional, targeted, efficient, and biocompatible imaging agents. Other classes of organic that absorb in the NIR range include bodipy dyes and trianionic 1,3,2-dioxaborine-containing polymethines.

### Methods and Materials

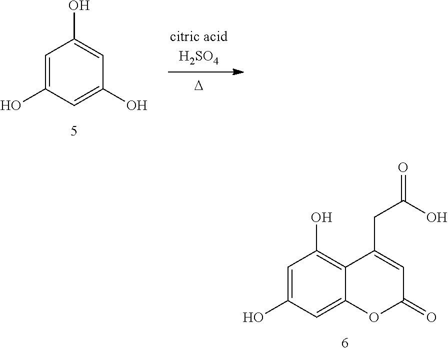

5,7-Dihydroxy-2-oxo-2H-1-benzopyran-4-acetic acid 6 Citric acid monohydrate (12.6 g, 60 mmol) was added portion-wise to 10 mL of vigorously stirring concentrated sulfuric acid followed by an additional 5 mL of concentrated sulfuric acid. The contents were gradually heated from room temperature to 70° C. over the course of 2 hours, then cooled in an ice-water bath for 30 minutes. Phloroglucinol dihydrate 5 (6.5 g, 40 mmol) was added into the thick medium, then rinsed down with an additional 5 mL of concentrated sulfuric acid. The flask was cooled in an ice-bath for 3 hours, then poured onto 150 mL of crushed ice. The solid was collected via filtration and washed with ice-cold deionized water. The white solid was dried over P₂O₅ in a vacuum desiccator overnight to isolate 4.2 g (44%) of the title compound.

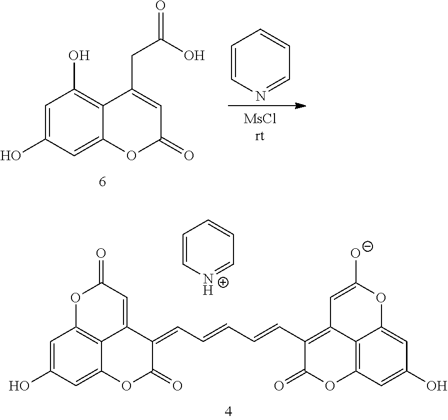 [SMILES: O=C(O)Cc1cc(=O)oc2cc(O)cc(O)c12 ; c1ccncc1 ; O=c1cc2/c(=C/C=C/C=C/c3c4c5c(cc(O)cc5oc3=O)OC([O-])=C4)c(=O)oc3cc(O)cc(o1)c32 ; c1cc[nH+]cc1]

Dye 4. A solution of acid 6 (236 mg, 1.00 mmol) in 2 mL of pyridine was prepared under a nitrogen atmosphere and treated with methanesulfonyl chloride (155 μL, 2.00 mmol) added dropwise. The reaction mixture was allowed to stand at room temperature for 1.5 h, then quenched with 20 mL of 0.25 M HCl. The dark precipitate was collected on a microporous filter, washed with 0.25 M HCl until the filtrate became colorless, then with deionized water and finally with diethyl ether. 136 mg (47% yield) of the product was collected after air drying.

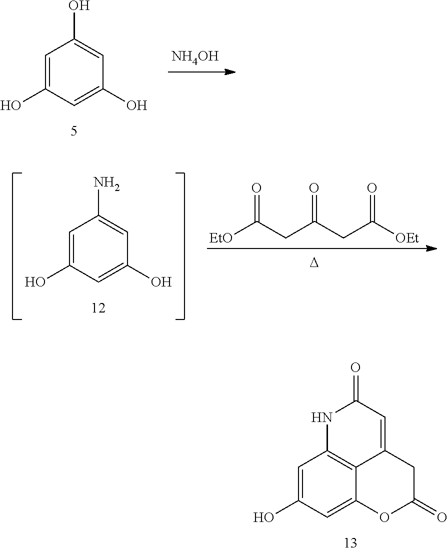

8-hydroxypyrano[4,3,2-de]quinoline-2,5(3H,6H)-dione (13) A flask charged with 5 (1.62 g, 10.0 mmol) and a stir bar was flushed with nitrogen, then 30 mL of degassed concentrated ammonium hydroxide was added via syringe. The reaction mixture was stirred at room temperature for 24 hours, then evaporated under reduced pressure. The flask with the resulting beige intermediate 12 (1.28 g crude weight) was equipped with a stir bar and an air-cooled condenser, flushed with nitrogen, and charged with diethyl acetone dicarboxylate (9 mL, 50 mmol) added via syringe. The flask was heated in an oil bath at 140° C. for 1 hour and allowed to cool to room temperature. The rust-colored solid was suspended in 10 mL of ice-cold ethanol, filtered off and rinsed on the filter with 2×10 mL ice-cold ethanol and 10 mL of diethyl ether. After drying, 1.35 g (62% yield) of the tan solid 13 was obtained.

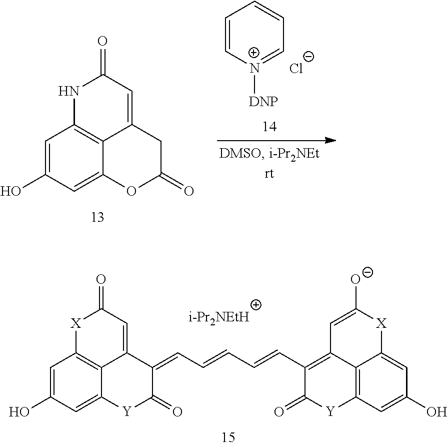

Dye 15. A solution of compounds 13 (218 mg, 1.00 mmol) and 14 (170 mg, 0.600 mmol) in 2 mL of dimethyl sulfoxide stirred under nitrogen was treated with Hünig's base (200 μL, 1.15 mmol) added dropwise via syringe. The reaction mixture was stirred at room temperature for 1.5 hours, then quenched with 20 mL of 0.25 M HCl. The solid was collected on a microporous filter, washed with 0.25 M HCl until the filtrate became colorless, then with deionized water followed by diethyl ether. Isolation afforded 263 mg of 4 as a crude mixture of isomers containing the 2,4-dinitroaniline by-product, which could be removed by additional washing with a 1:1 ethanol/water mixture until the filtrate became colorless.

### Spectroscopy

Absorption spectra were recorded on a Beckman Coulter DU 640 UV-visible spectrophotometer (Brea, CA) and fluorescence spectra were recorded on a Fluorolog-3 spectrofluorometer (Horiba Jobin Yvon, Inc., Edison, NJ). All fluorescence measurements were conducted at room temperature and were recorded at excitation of 700 nm and emission scan from 715-900 nm.

Fluorescence lifetimes (FLT) were measured using time-correlated single photon counting (TCSPC implemented in Fluorolog-3, Horiba Jobin Yvon) with a 700 nm excitation source NanoLed® (impulse repetition rate 1 MHz) at 90° to the PMT R928P detector (Hamamatsu Photonics, Japan). The dyes were dissolved in DMSO and the absorbance of the measured solutions was maintained below 0.15 at a 700 nm excitation wavelength. We chose DMSO as the solvent for this study because it provides a similar environment to the biological systems. The detector was set to 740 nm with a 20 nm bandpass and data were collected until the peak signal reached 10,000 counts.

### Cell Culture and Fluorescence Imaging

SKBR3-GFP-Luc cells (Dual labeled stable SK-BR-3 expressing green fluorescent protein (GFP) and luciferase, SL032, GeneCopoeia, Rockville, MD) were cultured in Dulbecco's Modified Eagle Medium (DMEM high glucose, Gibco) supplemented with 10% Fetal Bovine Serum (FBS), 1% penicillin/streptomycin and 5 μg/mL puromycin, and maintained in a humidified incubator at 37° C. with 5% CO₂ atmosphere. For imaging experiments, these GFP labeled HER2+ human breast cancer cells were plated in Fluorobrite DMEM media (Gibco) supplemented with 10% FBS and 1% pen/strep in an 8-well chamber slide (6.0×10⁴ cells in 200 μL media per well). After 24 h incubation (37° C., 5% CO₂), each NIR dye was added to the media diluted to a final concentration of 1 μM or 5 μM as noted with gentle mixing upon addition to each well of cells. The dyes were incubated with the live cells for 4 or 24 hr before aspirating the dye-containing media. The cells were then washed briefly with PBS (300 μL×2), and fixed with 4% paraformaldehyde (pH 7.1, 400 μL in PBS) for 15 min at room temp. Nuclei were then DAPI stained (200 μL, 1.4 μM in PBS) by incubating the fixed cells for 10 min at room temp. All cells were washed with DPBS (300 μL×2) before mounting (Fluoromount-G, #1 coverslip) and imaging the slides with either a Zeiss AxioImager Z2 fluorescence microscope or FLIM.

Fluorescence intensity imaging was conducted on a Zeiss Axioimager.Z2 microscope equipped with a Hamamatsu ORCA Flash 4.0 V2 camera, X-Cite Xylis LED-based light source, and 20× Zeiss Plan-Apochromat air objective (0.8 N.A.) The imaging parameters included DAPI: excitation 335-383 nm/emission 420-470 nm/exposure time 18 ns GFP: ex 450-490 nm/500-550 nm/46 ms exposure and NIR: ex 715-752 nm/760-855 nm/1 s exposure. Analysis was conducted in Zeiss Zen Blue software (version 3.6) and the contrast scaling was matched for each imaging channel.

### FLIM

Fluorescence Lifetime Microscopy (FLIM) was conducted on a PicoQuant MicroTime200 confocal microscope with TCSPC capabilities for detecting and timing photon events. Imaging was conducted on fixed SKBR3-GFP-Luc cells treated with 5 μM of compound 15 as described above using a 60× water immersion objective (Olympus UPlanApo 60×W, 1.2 NA).

Imaging parameters included excitation with a 785 nm laser oscillated at 10 MHz and 785 nm long pass emission. The image was acquired by recording the time delay between the excitation pulse and the emission photon at each pixel over an 80×80 μm field of view with 350×350 px resolution and 5.0 ms dwell time (12 min acquisition) to achieve optimal image quality with sufficient lifetime resolution.

Image and data analysis was performed using FLIM software SymPhoTime 64 version 2.7 (PicoQuant, Berlin, Germany). The FLIM image was constructed based on the average photon arrival times after the laser pulse at each pixel. The color scale represents the distribution of fluorescence lifetimes (FLT) across the image while the brightness encodes the fluorescence intensity. The FLT value was determined by fitting the overall image to a 2-exponential decay model with the n-exponential reconvolution method and predicted instrument response function for the 780 nm laser. The observed fluorescence lifetime components, percent contribution, and chi-squared to assess goodness-of-fit are reported.

### In-Vivo Fluorescence Imaging

Female athymic nude mice (Hsd:Athymic Nude-Foxn1nu) were acquired from Envigo (Inotiv, Indianapolis, IN) The mice were fed ad libitum and switched to low fluorescence chow at least 4 days before fluorescence imaging. Whole-body planar fluorescence imaging was performed on an IVIS Spectrum CT (Perkin Elmer) with 19 different excitation/emission pairs across the visible and NIR range to enable hyperspectral analysis. Following IV injection with 6 nmol of dye (100 μL, 60 μM in PBS), the live mice (n=2 per compound) were anesthetized with 2% isoflurane vaporized in oxygen and imaged at the noted time points (30 min, 2 h, 4 h, 24 h, and 48 h post-injection). The anesthetized mice were euthanized by cervical dislocation and ex vivo biodistribution studies were performed after imaging at the 48 h time point. Key organs (heart, lung, kidney, spleen, muscle, liver, skin, femur, brain, and blood) were then dissected and imaged on the IVIS Spectrum CT. All animal studies were performed in compliance with the Washington University School of Medicine Institutional Animal Care and Use Committee and in accordance with humane care and use of research animals. Image analysis was performed with Living Image (Perkin Elmer, version 4.8.0) and hyperspectral image analysis with IDCube software.

### Results

### Synthesis of Oxonol Type Dyes Open New Class of NIR Compounds

In the search for negatively charged NIR fluorophores, a new class of compounds was prepared that are structurally similar to oxazoles but emit at longer wavelengths. We rationalized that adding conjugation from four methines, such as in the compound to six methines would shift the absorption and emission roughly about 200 nm (100 nm per each added methine group) according to the Kuhn principle.

The example of the synthesis is shown in 4, which is readily produced in two steps. First, 1,3,5-trihydroxybenzene reacts in the presence of sulfuric acid. The resulting coumarin-4-acetic acid 6 is treated with methanesulfonyl chloride in pyridine to produce the dye 4 (Scheme 1).

To the best of our knowledge the synthesis of this compound has never been reported. The suggested mechanism of this reaction is shown in Scheme 2.

Similar products were obtained when preformed lactone 7 is subjected to the same conditions. Lactones similar to 7, which we term 1,6-dioxaphenalene-2,5-diones (DOPD), have been briefly reported in the literature. To the best of our knowledge, however, their chemistry remains virtually unexplored. It may be noted that linking two DOPD moieties with a 5-carbon conjugated chain results in a more extended π-system than those found in the known oxonol NIRFs 2 and 3. As a matter of fact, structure 3 may be thought of as two coumarins linked at the γ-position via a heptamethine chain, with additional lactone rings helping to enforce its planarity.

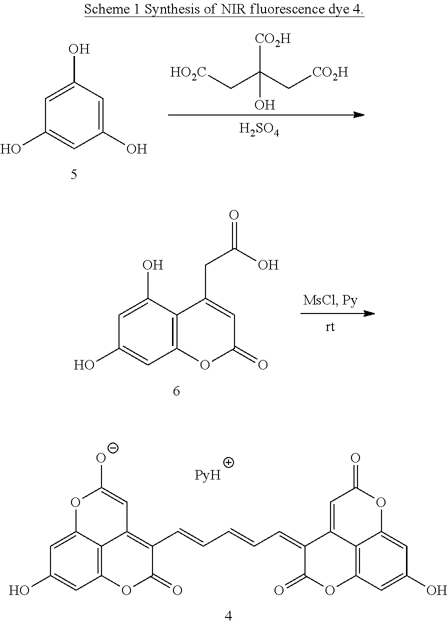

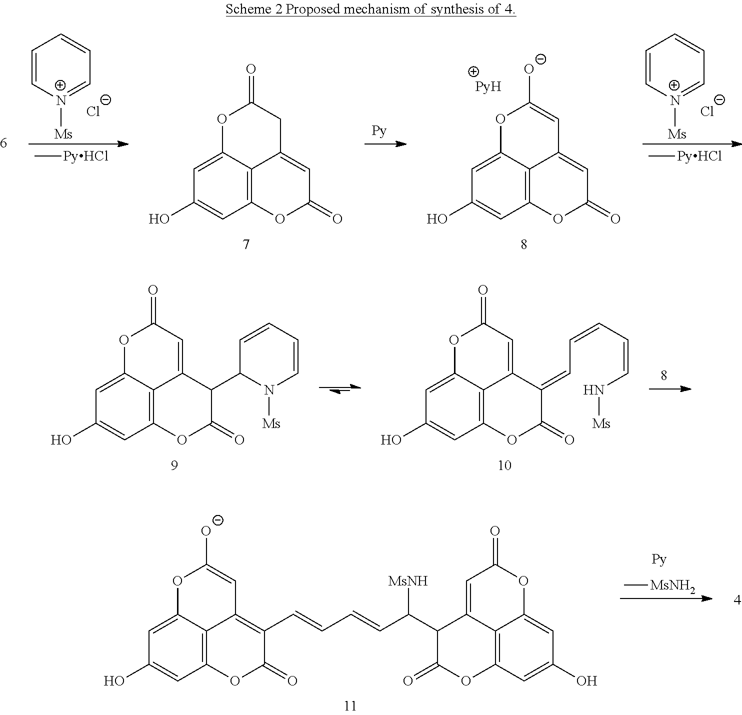

Structural modifications of the new chromophore can be introduced via the changes in the DOPD structure. To demonstrate the proof-of principle we replaced one of the oxygen atoms in the DOPD with a nitrogen. This approach led to the successful synthesis of a novel carbostyril-derived lactone 13 via a catalyst-free double condensation of 5-aminoresorcinol 12 with acetonedicarboxylate esters. While the original mesyl chloride-based procedure failed to lead to the product, the desired transformation, however, was achieved using a known Zincke salt 14. The resulting dye 15 was produced (depending upon the embodiment, as a mixture or single regioisomer) and used without purification.

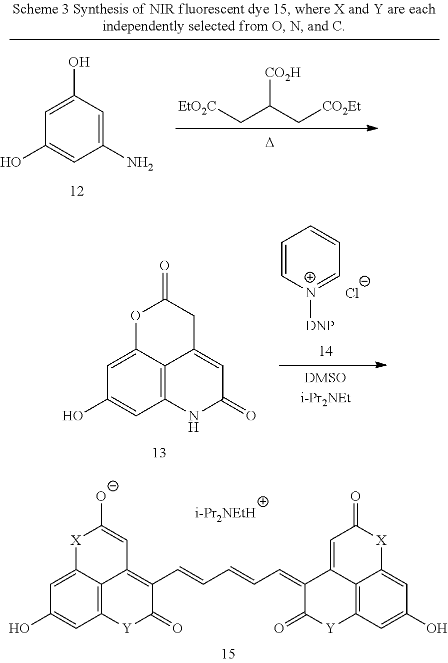

### Optical Properties of the NIR Oxazol-Type Fluorescent Dyes

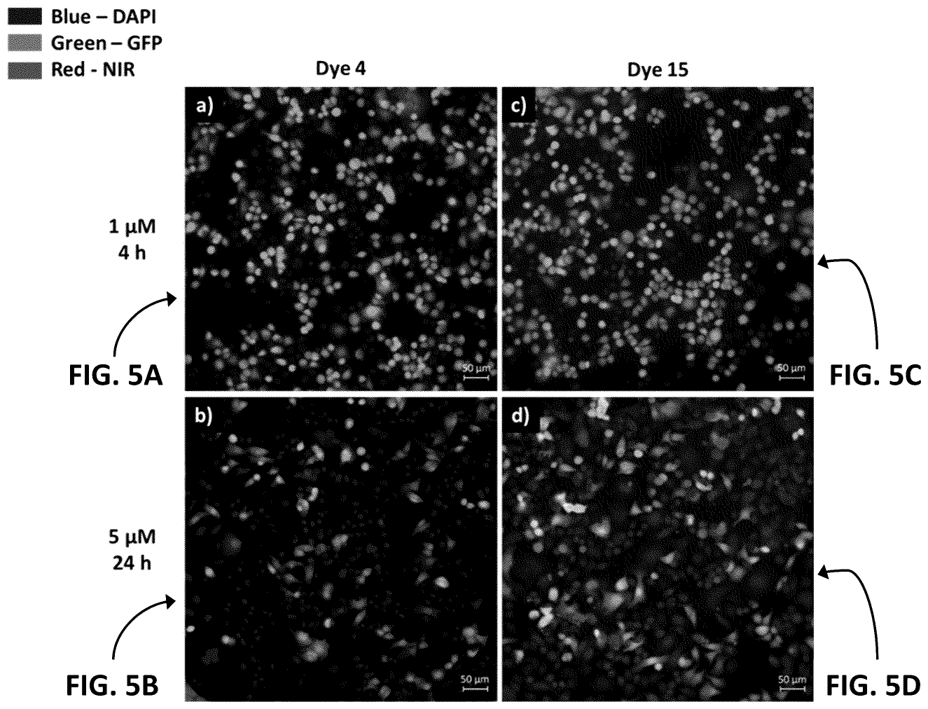

Spectral properties of the newly synthesized dyes 4 and 15 are summarized in Table 1. The compounds are relatively hydrophobic and have low solubility in water, their optical properties were measured in methanol and DMSO. The absorption spectra of 4 and 15 were measured in both methanol (FIG. 4(A-B)) and DMSO (FIG. 5(A-D)). The UV-Vis absorption spectra of both derivatives displayed a typical profile for absorption and emission spectra caynine dyes emitting in the same range with a relatively short Stoke's shifts of 10-15 nm. The broad emission bands allow flexibility in the choice of excitation and fluorescence wavelengths in analytical and biological assays. The relative fluorescence quantum yields of dyes 4 and 15 measured in DMSO using ICG as a standard show the values of 0.24 and 0.093 respectively compared to 0.12 for ICG.

TABLE 1
Summary of spectral properties of 4 and 15.

| | #4 | | #15 | |
|---|---|---|---|---|
| | Methanol | DMSO | Methanol | DMSO |
| Abs max, nm | 704 | 719 | 709 | 721 |
| Emission max, | 731 | 744 | 738 | 749 |
| nm | | | | |
| Fluorescent | 0.28 | 0.65 | 0.23 | 0.47 |
| lifetime, ns | | | | |
| Quantum yield | — | 0.24 | — | 0.09 |

Subsequently, the solvatochromism of 4 and 15 was investigated using absorption in DMSO, water and methanol with varying polarity and hydrogen bonding capabilities (FIG. 4(A-B)).

### Testing Dyes in Cellular Fluorescence Imaging

Out of the two dyes tested in cellulo, only compound 15 showed substantial cellular internalization in the human breast cancer cells after 24 hours of incubation as judged by the signal intensity in the NIR channel (FIG. 5(A-D)). This dye co-localizes with the GFP signal expressed in the cytosol in this cell line. Little overlap of this dye with DAPI staining of the nuclei suggests that dye 15 is predominantly localized to the cytoplasm once taken up by the cancer cells. This uptake is both time and concentration-dependent with limited fluorescent signal after incubation with a lower concentration (1 μM) of dye 15 for 4 h (FIG. 5C) compared with the longer and more concentrated dye incubation shown in FIG. 5D. The intracellular uptake and fluorescence of dye 15 indicates this new class of dyes has promise for biological imaging applications in the NIR range, which is highly advantageous due to reduced autofluorescence as well as increased tissue penetration depth for in vivo use.

Interestingly, dye 4 which has a relatively small structural change to ketones in place of imines in dye 15 has essentially no appreciable uptake in this cancer cell line even at a relatively high concentration and incubation time (5 μM, 24 h). This is a dramatic change in behavior in its interaction with cells suggesting this fluorophore scaffold may be highly tunable through chemical modification. Given the very limited non-specific cellular uptake of dye 4 while having a similar fluorescence emission spectrum to 15 as determined through in vitro spectroscopy, this dye has excellent potential for the development of a conjugatable version to use in novel targeted NIR optical molecular imaging agents. Ensuring molecular specificity of the observed signal is crucial for these types of targeted agents so avoiding non-specific cellular uptake of the free dye is a necessary prerequisite.

### Fluorescence Lifetime Imaging of Probe 15 in Cells

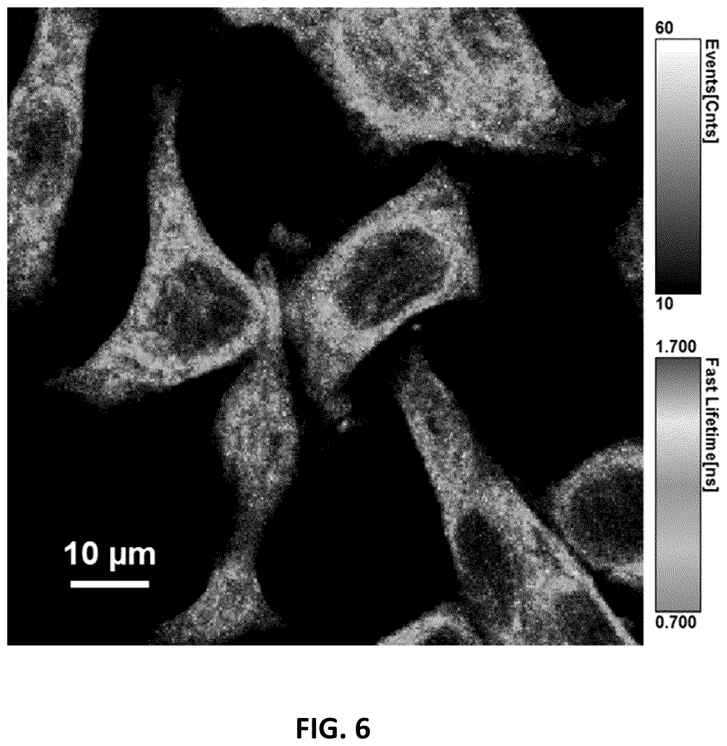

Fluorescence lifetime (FLT) imaging is sensitive to immediate environment of fluorescent materials and can reveal potential variations in the molecular environment, interactions, or conformational changes of the fluorophores. FLIM of SKBR3 breast cancer cells treated with dye 15 revealed a generally consistent FLT of this dye across the intracellular environment (FIG. 6). This further suggests that dye 15 is taken up in the cytoplasm non-specifically rather than internalized into endosomes or lysosomes through a receptor-mediated pathway. Additionally, the higher magnification shows central regions of these cells lacking NIR fluorescence signal which further supports our finding that dye 15 is not localized to the nuclei of these cells.

In conclusion, new oxonol NIRFs have been developed herein featuring previously unexplored diheterophenalenedione (DHPD) end groups.

### DISCUSSION

The shift from cationic to anionic NIR fluorescent dyes represents a significant change in the interaction dynamics between the dye and biological systems. Most existing NIR fluorescent dyes are cationic, which influences their binding affinity to cellular and molecular structures within biological tissues. This is because cell membranes are generally composed of lipid bilayers that present negatively charged surfaces externally, due to the presence of phospholipids with phosphate heads. Cationic dyes, with their positive charge, are naturally attracted to these negative charges on the cell surfaces, facilitating their binding and uptake into cells.

Anionic NIR fluorescent dyes, however, alter this interaction mechanism due to the negative charge of the dye. The repulsive forces between the negatively charged dye and the cell membranes lead to different binding affinities and uptake rates compared to cationic dyes, thus affecting the efficiency of cellular labeling and imaging applications where intracellular accumulation of the dye is desired. In embodiments of the present disclosure, the anionic dye preferentially binds to positively charged biomolecules, such as certain proteins, peptides, or amino acids, within the extracellular matrix or on the cell surface and are then carried via specific transporters inside the cell. Consequently, the charge of the dye significantly influences its biodistribution, retention time in the body, and clearance mechanisms. Anionic dyes show different patterns of distribution and clearance compared to cationic dyes, therefore affecting the dye's utility for long-term imaging studies depending upon the embodiment. Finally, the charge on the dye also influences its biocompatibility and toxicity. Anionic dyes interact differently with cellular components, leading to reduced toxicity in embodiments where they are less prone to penetrate cell membranes.

Opportunities for Novel Applications: The unique properties of an anionic NIR fluorescent dye enables applications not well-suited to cationic dyes, such as targeting specific cellular or tissue types based on their unique biochemical environments or developing new diagnostic tools that exploit the differential interactions of anionic dyes with biological tissues.

In summary, transitioning from cationic to anionic NIR fluorescent dyes introduces a range of potential changes in biological interactions, affecting everything from cellular uptake to biodistribution, targeting specificity, and safety profiles. Understanding and optimizing these properties will be crucial for harnessing the full potential of anionic NIR dyes in biological imaging and diagnostic applications.

What is claimed is:

1. An imaging agent composition comprising at least one compound according to 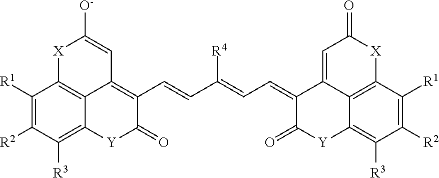
Formula (I) or resonance structure, tautomer, protonated version, or pharmaceutically acceptable salt thereof;
wherein X and Y are independently selected from 0 and N—R⁵; and
wherein R¹, R², R³, R⁴, and R⁵ are independently selected from H, alkyl, alkenyl, alkynyl, aryl, acyl, carboxy, carbalkoxy, carbamoyl, halogen, hydroxy, alkoxy, acyloxy, sulfonyloxy, alkylamino, dialkylamino, acylamino, sulfonylamino, sulfhydryl, and alkylthio.

2. The imaging agent composition of claim 1, wherein X is O, and Y is O.

3. The imaging agent composition of claim 1, wherein X is O, and Y is N—R⁵.

4. The imaging agent composition of claim 1, wherein X is N—R⁵, and Y is O.

5. The imaging agent composition of claim 1, wherein X is N—R⁵, and Y is N—R⁵.

6. A method of imaging a subject, the method comprising:
administering to the subject an imaging agent composition comprising a compound according to 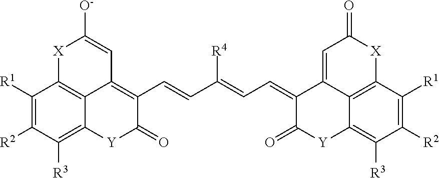
Formula (I) or resonance structure, tautomer, protonated version, or pharmaceutically acceptable salt thereof;
wherein X and Y are independently selected from O and N—R⁵;
wherein R¹, R², R³, R⁴, and R⁵ are independently selected from H, alkyl, alkenyl, alkynyl, aryl, acyl, carboxy, carbalkoxy, carbamoyl, halogen, hydroxy, alkoxy, acyloxy, sulfonyloxy, alkylamino, dialkylamino, acylamino, sulfonylamino, sulfhydryl, and alkylthio; and
applying an imaging modality to the subject.

7. The method of claim 6, wherein X is O, and Y is O.

8. The method of claim 6, wherein X is O, and Y is N—R⁵.

9. The method of claim 6, wherein X is N—R⁵, and Y is O.

10. The method of claim 6, wherein X is N—R⁵, and Y is N—R⁵.

11. The method of claim 6, wherein the imaging modality is a fluorescence-based optical imaging modality.

12. The method of claim 11, wherein the imaging modality is selected from single photon imaging, multiphoton imaging, fluorescence lifetime imaging (FLIM), and Foster Resonance Energy Transfer (FRET).

13. An imaging agent precursor composition comprising
a 1,6-diheterophenalene-2,5-dione according to 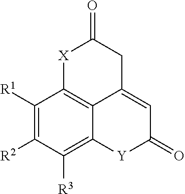
Formula (II) or resonance structure, tautomer, protonated version, or pharmaceutically acceptable salt thereof;
wherein X and Y are independently selected from O and N—R⁵; and
wherein R¹, R², R³, and R⁵ are independently selected from H, alkyl, alkenyl, alkynyl, aryl, acyl, carboxy, carbalkoxy, carbamoyl, halogen, hydroxy, alkoxy, acyloxy, sulfonyloxy, alkylamino, dialkylamino, acylamino, sulfonylamino, sulfhydryl, and alkylthio.

14. The imaging agent precursor composition of claim 13, wherein X is O, and Y is O.

15. The imaging agent precursor composition of claim 13, wherein X is O, and Y is N—R⁵.

16. The imaging agent precursor composition of claim 15, wherein R⁵ is H.

17. The imaging agent precursor composition of claim 13, wherein X is N—R⁵, and Y is O.

18. The imaging agent precursor composition of claim 13, wherein X is N—R⁵, and Y is N—R⁵.

19. The imaging agent precursor composition of claim 13, wherein the 1,6-diheterophenalene-2,5-dione according to Formula (II) is selected from a coumarin-derived lactone and a coumarin-derived lactam.

20. The imaging agent precursor composition of claim 13, wherein the 1,6-diheterophenalene-2,5-dione according to Formula (II) is selected from a carbostyril-derived lactone and a carbostyril-derived lactam.
